(19) United States
(12) Patent Application Publication
(10) Pub. No.: US 2011/0100362 A1
(43) Pub. Date: May 5, 2011

(54) Pneumatic Shunt Resistance and Gas Mixer

(75) Inventors: Martin Baecke, Dessau (DE); Uwe Heimhalt, Kothen (DE); Hartung Peter, Halle/Saale (DE)

(73) Assignee: FRITZ STEPHAN GMBH, Gackenbach (DE)

(21) Appl. No.: 11/990,196

(22) PCT Filed: Aug. 2, 2006
(86) PCT No.: PCT/DE2006/001352

§ 371 Date: Apr. 21, 2010

(30) Foreign Application Priority Data
Aug. 11, 2005 (DE) 10 2005 038 349.1

(51) Int. Cl. A61M 16/10; E03B 7/07

(52) U.S. Cl. 128/203.12; 137/551

(57) ABSTRACT

This invention relates to pneumatic shunt resistance, comprising a block (2) having a gas-containing longitudinal cavity (4) intersected by a likewise longitudinal measurement cavity (6). The two cavities are arranged such that the measurement cavity (6) intersects the gas-containing cavity (4) so as to define a sectional region, wherein, in sections perpendicular to its longitudinal extension, the measurement cavity (6) has a similar cross-section in the sectional region of the two cavities. The pneumatic shunt resistance moreover comprises a resistor (13) located in the sectional region of the two cavities and having a shape similar to that of the measurement cavity (6) at this point, so that a gap remains between the wall of the resistor (13) and the wall of the measurement cavity. Furthermore, this invention relates to gas mixers having two flow control loops and one block, thereby reducing the number of tubes.

The invention relates to the technical field of gas mixers. Specifically, the invention relates to gas mixers used for the artificial respiration of patients.

As a rule, gas mixers conventionally used in the field of intensive medicine are based on complex mechanical pressure controllers (Dräger, and others), which are adjusted by means of mechanical rotary switches. Alone an adjustment error thereof is frequently greater than the required accuracy in the gas mixture.

Flow controllers as a combination of gas flow sensor and proportional valve are known and customary in the trade. Gas mixers or gas dosing devices as parallel connection of flow controllers are known as discrete assemblies.

It is the object of the invention to provide a compact pneumatic shunt resistance and a compact gas mixer.

This object is achieved with the subject matters of the independent claims.

Preferred embodiments of the invention are defined in the dependent claims.

An advantage of a flow through a gap is that the flow is laminar. This results in a characteristic curve which is linear over large regions.

An advantage of the use of a gap between a cylindrical measurement cavity and a cylindrical resistor is the easy manufacturability, for example, by drilling, injection molding or turning.

A gap of a uniform width advantageously permits a large linear region. Advantageously, this is achieved with the coincidence of the rotational axes of the resistor and the measurement cavity.

If the resistor is part of an insert, which is screwed into the block by means of a thread, the flow resistance of the shunt resistance can be readily adapted to the requirements by different inserts.

An O-ring provides for the pressure tightness in an easy manner.

A gas mixer comprising a block with the most important pneumatic connections and to which as many valves and sensors as possible are directly fixed and pneumatically connected requires little space and is easy to mount because the number of tubes is reduced as fewer tubes are required.

Advantageously, a pneumatic shunt resistance according to the invention can be integrated in the block of the gas mixer, thereby simplifying the adaptation of the gas mixer to gas sources which provide different pressures.

A sword insert provides for a fast and complete mixing of the gases to be mixed. This is particularly advantageous if gas sensors are used, so as to avoid faulty measurements.

The constriction together with a storage volume, which is connectable to the connection connected in parallel to the gas outlet, forms a pneumatic low pass, which increases the stability of the control loops, even if different amounts of gas are delivered at the outlet during the inspiration and the expiration.

Advantageously, an oxygen sensor can be calibrated with the aid of the synthetic air supplied thereto, and it can verify the oxygen concentration of the mixed gas upon switching over a corresponding switch valve.

The use of additional blocks advantageously permits the adaptation of the gas mixer to different requirements. By using like component parts in different apparatus the manufacturing costs can thus be reduced due to the larger scale manufacture.

With the aid of the second additional block it is advantageously possible to provide the upstream outlets with a specific pressure level from the basic block and to adjust a pressure or throughput level for the respirator connection separately.

A preferred embodiment of the invention will be explained in more detail below by means of the accompanying drawings. In the drawings:

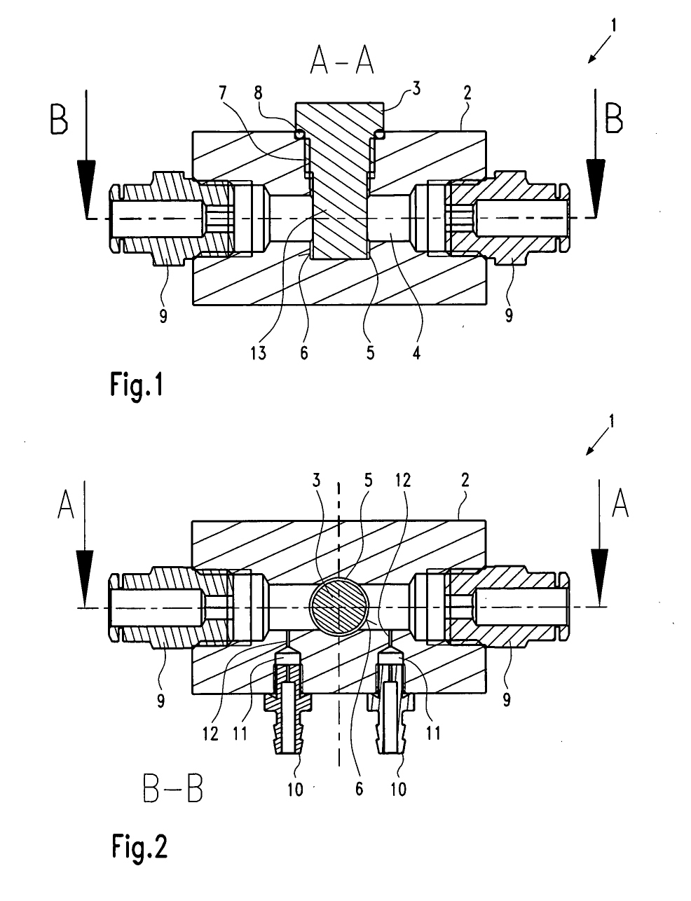

FIG. 1 shows a vertical section through a pneumatic shunt resistance according to the invention;

FIG. 2 shows a horizontal section through a pneumatic shunt resistance according to the invention;

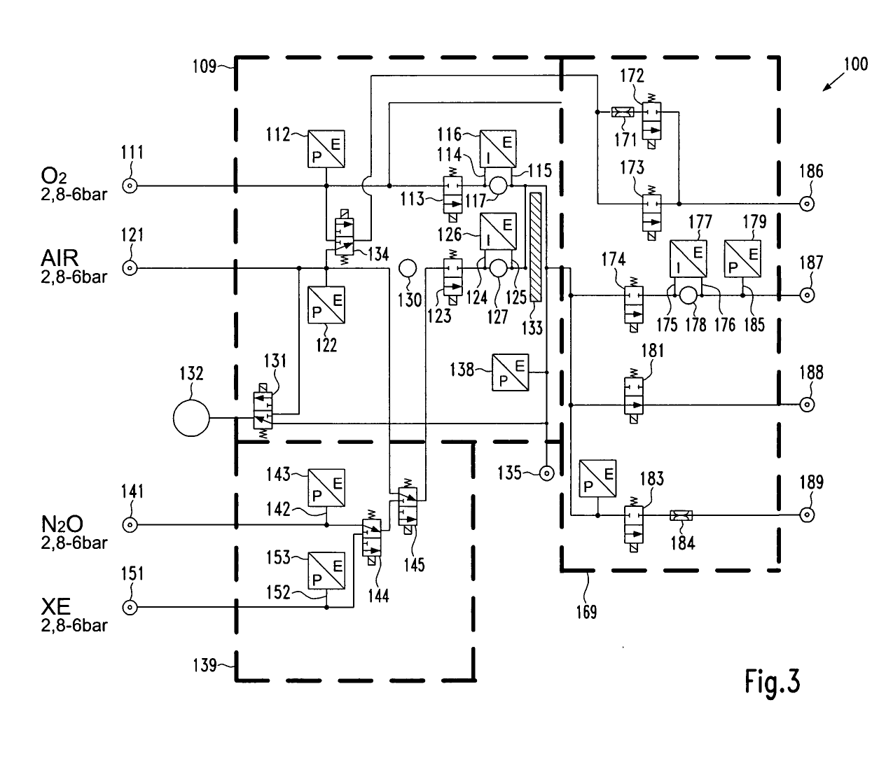

FIG. 3 shows a pneumatic circuit diagram of the gas mixer according to the invention;

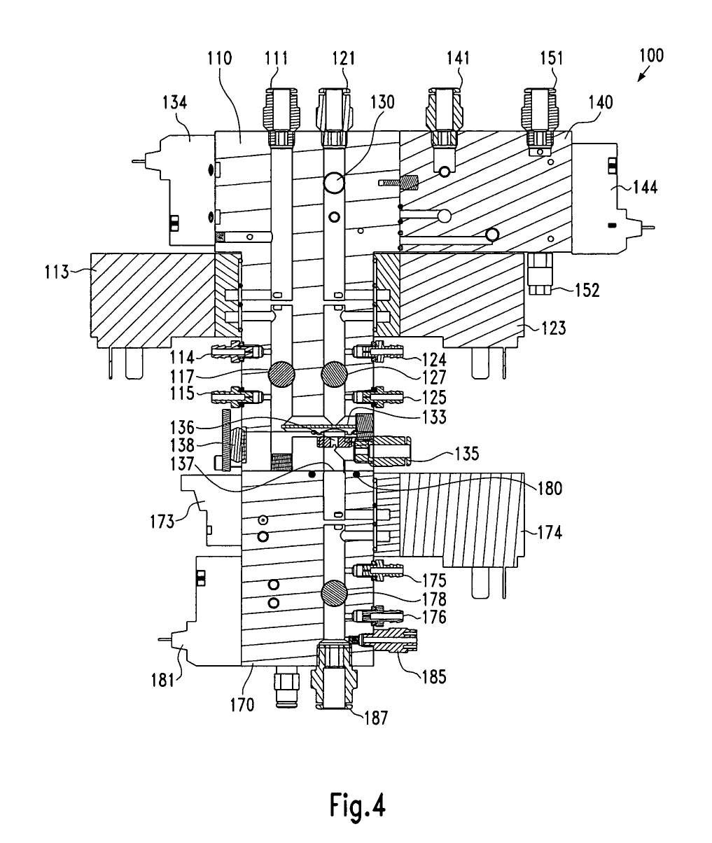

FIG. 4 shows a section through the gas mixer according to the invention; and

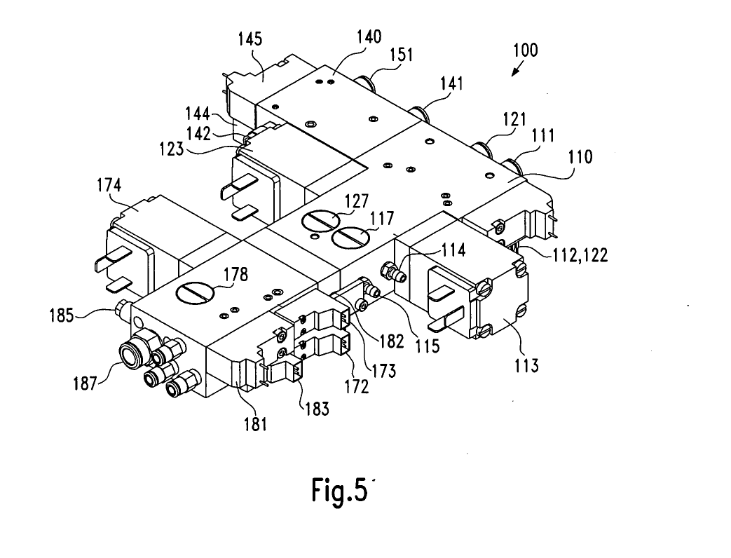

FIG. 5 shows a perspective view of the gas mixer according to the invention.

FIGS. 1 and 2 show two sections through a pneumatic shunt resistance 1 according to the invention. In a block 2 there are located a gas-containing bore 4, on the left and right respectively a tube connection 9 on the gas-containing bore 4, two sensor connections 10 as well as an insert 3. The insert 3 is screwed into block 2 by a thread 7.

In the section illustrated in FIG. 1, the insert 3 comprises in its lower portion a cylinder 13. The cylinder 13 is situated in a measurement bore 6 perpendicularly crossing the gas-containing bore, whereby also the center lines of both bores are crossing each other. The diameter of the measurement bore 6 is slightly larger than the diameter of the gas-containing bore 4. A narrow gap 5 of some tenths of millimeters is formed between the measurement bore 6 and the cylinder 13. In the gap 5 the flowing gas is caused to flow in an approximately laminar manner so as to obtain a monotonous, region-wise linear characteristic curve for the interrelationship of pressure drop and flow. The insert 3 has a sealing collar, which is sealed against block 2 by an O-ring 8. Such inserts are easy to manufacture.

As compared to other flow resistances, the pneumatic shunt resistance according to the invention allows the formation of a laminar flow in a very easy manner. By replacing the insert 3 by another insert having a cylinder 13 with a slightly different radius the dimension of gap 5 can be adapted to the flow range, so that a high variability is obtained without having to make alterations to block 2.

The basic adaptation is accomplished by the choice of the measurement bore 6 relative to the gas-containing bore 4.

The pressure drop can be sensed at the measurement bore 6 through cross holes 12, sensor connection bores 11 and sensor connections 10. To the sensor connections 10 either a flow sensor or a differential pressure sensor may be connected, which extends the pneumatic shunt resistance 1 to a flow sensor.

FIGS. 3, 4 and 5 show different embodiments of a gas mixer according to the invention. FIG. 3 shows a pneumatic circuit diagram of the fully equipped gas mixer 100 according to the invention. FIG. 4 shows a section through an embodiment of such a gas mixer 100, which is illustrated in a perspective view in FIG. 5.

The gas mixer comprises a basic module 109, a first additional module 139 as well as a second additional module 169. The core pieces of the modules are comprised of a basic block 110, a first additional block 140 and a second additional block 170, respectively. The implementation in milled and drilled blocks, e.g. of anodized aluminum, guarantees a compact construction. The required but non-illustrated circuit board is situated in parallel with respect to and directly below the blocks. The assembly time is significantly reduced as compared to discretely constructed gas mixers because the gas mixer according to the invention requires fewer tubes for connecting the required valves and sensors by tubes.

In the simplest embodiment the gas mixer is comprised of a basic module in which the mixing of two gas flows is realized.

The oxygen connection 111 is, as the name implies, provided for the connection to oxygen. At air connection 121 artificial air or air compressed by a compressor is supplied. In the embodiment shown in FIGS. 4 and 5 the supply of gases is accomplished through tubes. Therefore, tube connections are illustrated at the connections in FIGS. 4 and 5. In another embodiment, for example, a non-illustrated filter module can be directly attached to the basic block 110 and sealed with respect to the same.

Each of the two gas flows first passes through a pressure sensor 112 and 122, respectively, in which the pressure on the oxygen connection 111 and the air connection 121, respectively is measured. Then, each gas flow passes through a combination of a proportional valve 113 and 123, respectively, and a gas flow sensor 114, 115, 116, 117 and 124, 125, 126, 127, respectively. Each gas flow sensor is comprised of a pneumatic shunt resistance as was explained by means of FIGS. 1 and 2. FIGS. 3 to 5 show, by way of example, the inserts 117 and 127, respectively. Flow sensors 116 and 126, respectively, are connected by the flow sensor outlets and inlets 114 and 115 and 124 and 125, respectively. As was mentioned above, also differential pressure sensors may be used in lieu of the flow sensors 116 and 126. A non-illustrated prior electronic circuit connects the two proportional valves to the two gas flow sensors to form two control loops, whereby in each control loop the position of the proportional valve is adjusted in such a way that a predetermined gas flow is possibly maintained.

Next, the two gas flows are mixed in the cross hole, in which a sword insert 133 is provided. To achieve a good and fast mixing, the sword insert 133 causes the gas coming from the air connection 121 to flow first to the left in the upper half (compare FIG. 4) of the cross hole where it encounters the gas coming from the oxygen connection, and to flow then through the lower half of the cross hole toward the constriction 136 and further to the gas outlet 137 of the basic module.

If two gas flows each having a constant flow are joined, a new gas flow likewise having a constant flow is created. Alternatively it is also possible to adjust a specific target pressure at the gas outlet 137 by means of a pressure sensor 138 mounted at the mixing location.

By a connection 135 a non-illustrated compensation reservoir may be connected so that the constriction 136 and the compensation reservoir form a pneumatic low pass. This low pass reduces cross interferences affecting the control loops if, for example, during the inspiration and expiration different amounts of gas are delivered at the gas outlet 137.

The valves for the airway and the inserts 117 and 127 may be exchanged depending on the case of application, thereby permitting an optimum control of both the supply of compressed air from the bottle or the hospital system (2-7 bar) and poorly compressed air from a compressor (150 mbar).

On the inlet side a first additional module 139 may be mounted, by means of which, as an alternative to air, up to two other gases may be connected simultaneously, e.g. nitrous oxide at the nitrous oxide connection 141 or inert gas such as helium or xenon at the inert gas connection 151. By means of the pressure sensors 143 and 153, respectively, which are connected by the connections 142 and 152, respectively, the input pressure may be verified, for example, to indicate an empty gas bottle by means of an alarm. A series connection of switch valves 144 and 145 makes sure that always only one of the three gases is mixed with oxygen. If the first additional module 139 is connected, the airway is interrupted by an interrupter 130 to avoid a pneumatic short-circuit of the switch valve 145. If the first additional module 139 is not necessary, the connection openings in the basic block are tightly sealed.

Instead of the first additional module 139 a valve 131 and a connection for an oxygen sensor 132 may be mounted on the basic block 110. Merely because of the dimensions of the components is it impossible in the currently considered embodiment of basic block 110 to mount both the valve 131 and the first additional module 139 simultaneously. This is why FIG. 3 shows the combination of both variants. In the one position of valve 131 the oxygen sensor 132 can be calibrated either by means of the gas supplied at the air connection 121 or measure the oxygen concentration of this gas, or it can monitor the oxygen concentration of the gas mixture delivered at the gas outlet 137 if the valve 131 is in the other position shown in FIG. 3. Specifically due to this analyzing possibility it is necessary that the two gas streams be mixed fast. Otherwise, the measured oxygen concentration also depends on the branch-off point of the return line to valve 131, which is not acceptable. This return line is branched off upwardly out of the plane of the drawing just in front of the constriction 136 so that it is not illustrated in FIG. 4.

A second additional module 169 with the second additional block 170 can be connected on the outlet side. The second additional module 169, again, includes a gas flow sensor comprising the pneumatic shunt resistance according to the invention with insert 178, a flow sensor outlet 175 and a flow sensor inlet 176, to which a flow sensor 177 may be connected, a proportional valve 174 and a connection 185 for a pressure sensor 179 to allow the adjustment of the flow to or the pressure at the respirator connection 187 independently of the flow through and pressure at the gas outlet 137.

Besides, the second additional module 169 has an injector connection 186, which may directly be supplied with gas from the oxygen connection 111 or the air connection 121 via the valve 173 and the switch valve 134. Alternatively, the flow through the injector connection 186 can be limited by a valve 172 and a capillary 171. The capillary may have an opening, for example, of 0.3 mm.

Moreover, the second additional module 169 includes a hand-operated respirator connection 188, which may be connected by a valve 181 to the gas outlet 137. Finally, the second additional module 169 includes a nebulizer connection 189, which may connected by a capillary 184 and a valve 183 to the gas outlet 137. The capillary 184 may have an opening, for example, of 0.8 mm.

In one embodiment, the pressure sensors 112, 122, 138, 143, 153 have a measurement range of 0 to 7 bar. In this embodiment the pressure sensor 179 has a measurement range of 0 to 300 mbar. The valves 131, 172, 173 and 183 have a nominal width of 0.7 mm. The valves 134, 144, 145 and 181 have a nominal width of 1.5 mm.

The gas mixer was developed for the production of gas mixtures for respiration, but can be applied for plenty of other gas mixture functions.

The invention was explained in more detail by means of preferred embodiments above. A person skilled in the art will appreciate, however, that various alterations and modifications may be made without departing from the spirit of the invention. Therefore, the scope of protection will be defined by the accompanying claims and their equivalents.

### LIST OF REFERENCE NUMBERS

1 pneumatic shunt resistance
2 block
3 insert
4 gas-containing bore
5 gap
6 measurement bore
7 thread
8 O-ring
9 tube connection
10 sensor connection
11 sensor connection bore
12 cross hole
13 cylinder
100 gas mixer
109 basic module
110 basic block
111 oxygen connection
112 pressure sensor
113 proportional valve
114 flow sensor outlet
115 flow sensor inlet
116 flow sensor
117 insert
121 air connection
122 pressure sensor
123 proportional valve
124 flow sensor outlet
125 flow sensor inlet
126 flow sensor
127 insert
130 interrupter
131 valve
132 oxygen sensor
133 sword insert
134 valve
135 connection
136 constriction
137 gas outlet
138 pressure sensor
139 first additional module
140 first additional block
141 nitrous oxide connection
142 connection
143 pressure sensor
144, 145 valve
151 inert gas connection
152 connection
153 pressure sensor
169 second additional module
170 second additional block
171 capillary
172, 173 valve
174 proportional valve
175 flow sensor outlet
176 flow sensor inlet
177 flow sensor
178 insert
179 pressure sensor
180 O-ring
181 valve
183 valve
184 capillary
185 connection
186 injector connection
187 respirator connection
188 hand-operated respirator connection
189 nebulizer connection

1. A pneumatic shunt resistance, comprising
a block (2) having a gas-containing longitudinal cavity (4) intersected by a likewise longitudinal measurement cavity (6), wherein the two cavities are arranged such that the measurement cavity (6) intersects the gas-containing cavity (4) so as to define a sectional region, wherein, in sections perpendicular to its longitudinal extension, the measurement cavity (6) has a similar cross-section in the sectional region of the two cavities; and
a resistor (13) located in the sectional region of the two cavities and having a shape similar to that of the measurement cavity (6) at this point, so that a gap remains between the wall of the resistor (13) and the wall of the measurement cavity.

2. The pneumatic shunt resistance according to claim 1, characterized in that both the measurement cavity (6) and the resistor (13) have a circular cross-section in the sectional region and are thus cylinders.

3. The pneumatic shunt resistance according to claim 2, characterized in that the axis of the resistor (13) coincides with the axis of the measurement cavity (6).

4. The pneumatic shunt resistance according to one of the preceding claims, characterized in that the resistor (13) is part of an insert (3; 117, 127, 178), wherein both the insert (3; 117, 127, 178) and the block (2) have a thread (7) so that the insert is retained in the block (2) by the thread (7).

5. The pneumatic shunt resistance according to claim 4, characterized in that the insert (3; 117, 127, 178) is sealed with respect to the block (2) by an O-ring.

6. A gas mixer, comprising
a block (110) having a plurality of cavities for the conduction of gas and being made of one piece, said block (110) comprising:
a first gas inlet (111);
a first connection for a pressure sensor (112) pneumatically connected to the first gas inlet (111);
a first flow sensor outlet (114);
a first flow sensor inlet (115);
a second gas inlet (121);
a second connection for a pressure sensor (122) pneumatically connected to the second gas inlet (121);
a second flow sensor outlet (124);
a second flow sensor inlet (125);
a gas outlet (137) pneumatically connected to the first and second flow sensor inlet (115, 125); said gas mixer further comprising:
a first proportional valve (113) mechanically connected to the block (110), the inlet of which is pneumatically connected to the first gas inlet (111) and the outlet of which is pneumatically connected to the first flow sensor outlet (114); and
a second proportional valve (123) mechanically connected to the block (110), the inlet of which is pneumatically connected to the second gas inlet (121) and the outlet of which is pneumatically connected to the second flow sensor outlet (124).

7. The gas mixer according to claim 6, characterized in that the first flow sensor outlet (114) is pneumatically connected to the first flow sensor inlet (115) by a pneumatic shunt resistance according to one of claims 1 to 5 and the second flow sensor outlet (124) is pneumatically connected to the second flow sensor inlet (125) also by a pneumatic shunt resistance according to one of claims 1 to 5, wherein the block (110) of the gas mixer also forms the blocks of the pneumatic shunt resistances.

8. The gas mixer according to one of claim 6 or 7, characterized in that the block (110) has a first longitudinal cavity connecting the first flow sensor inlet (115) to the gas outlet (137), wherein the block (110) has a second longitudinal cavity extending in parallel to the first longitudinal cavity, wherein the block (110) has a third longitudinal cavity intersecting with the first and second longitudinal cavity, wherein a sword insert (133) projects into the third longitudinal cavity dividing the third longitudinal cavity along the longitudinal axis in two parts so that, during operation, gas flows from the first longitudinal cavity first through the first part of the third longitudinal cavity to the second longitudinal cavity, where it mixes with gas from the second longitudinal cavity, and through the second part of the third longitudinal cavity back to the first longitudinal cavity and through the first longitudinal cavity to the gas outlet (137).

9. The gas mixer according to one of claims 6 to 8, characterized in that the block (110) has a first longitudinal cavity connecting the first flow sensor inlet (115) to the gas outlet (137), wherein the first longitudinal cavity has a constriction (136), wherein a further cavity connects the part of the first longitudinal cavity between the constriction (136) to a connection (135).

10. The gas mixer according to one of claims 6 to 9, characterized in that a switch valve (131) is mechanically fixed to the block (110), which has a first and a second inlet, wherein the first inlet of the switch valve (13) is pneumatically connected to the second gas inlet (121), wherein the second inlet of the switch valve (131) is pneumatically connected to the gas outlet (137) and wherein the outlet of the switch valve (131) is pneumatically connectable to an oxygen sensor.

11. The gas mixer according to one of claims 6 to 10, characterized in that a first additional block (140) is mechanically fixed to the block (110), said additional block (140) comprising:
a third gas inlet (141);
a third connection (142) for a pressure sensor (143) pneumatically connected to the third gas inlet (141);
a fourth gas inlet (151);
a fourth connection (152) for a pressure sensor (153) pneumatically connected to the fourth gas inlet (151); wherein the following is mechanically fixed to the additional block (140):
a second switch valve (144) having two inlets, wherein the first inlet of the second switch valve (144) is pneumatically connected to the third gas inlet (141) and the second inlet of the second switch valve (144) is connected to the fourth gas inlet (151);
a third switch valve (145) having two inlets, wherein the first inlet of the third switch valve (145) is pneumatically connected to an outlet of the second switch valve (144) and the second inlet of the third switch valve (145) is pneumatically connected to the second gas inlet (121), wherein the outlet of the third switch valve (145) is pneumatically connected to the inlet of the second proportional valve (123); and
wherein an interrupter (130) is mounted in the block (110), so that the pneumatic connection between the second gas inlet (121) and the inlet of the second proportional valve (123) is interrupted.

12. The gas mixer according to one of claims 6 to 11, characterized in that a second additional block (170) is mechanically fixed to the block (110), wherein an inlet of the second additional block is pneumatically connected to the gas outlet (137), wherein a pressure sensor (138) is mechanically fixed to the block (110) on a fifth connection for a pressure sensor, wherein the fifth connection for a pressure sensor is pneumatically connected to the gas outlet (137), wherein the following is mechanically fixed to the second additional block (170):
a third proportional valve (174), the inlet of which is pneumatically connected to the inlet of the second additional block (170);
a third flow sensor outlet (175) pneumatically connected to the outlet of the third proportional valve (174);
a third flow sensor inlet (176);
a sixth connection (185) for a pressure sensor pneumatically connected to the third flow sensor inlet (176);
a respirator connection (187) pneumatically connected to the third flow sensor inlet (176);
a first valve (181), the inlet of which is pneumatically connected to the inlet of the second additional block (170) and the outlet of which is pneumatically connected to a hand-operated respirator connection (188);
a second valve (183), the inlet of which pneumatically connected to the inlet of the second additional block (170);
a capillary (184), the inlet of which is pneumatically connected to the outlet of the second valve (183) and the outlet of which is pneumatically connected to a nebulizer connection (189).
